(19) United States
(12) Patent Application Publication
(10) Pub. No.: US 2005/0209211 A1
(43) Pub. Date: Sep. 22, 2005

(54) Trihemihydrate, anhydrate and novel hydrate forms of Cefdinir

(76) Inventors: Devalina Law, Libertyville, IL (US); Rodger F. Henry, Wildwood, IL (US); Xiaochun Lou, Long Grove, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/072,568

(22) Filed: Mar. 3, 2005

Related U.S. Application Data
(60) Provisional application No. 60/553,643, filed on Mar. 16, 2004.

(51) Int. Cl. C07D 501/14; A61K 31/545

(52) U.S. Cl. 514/202; 540/222

(57) ABSTRACT

The present invention relates to trihemihydrate, novel lower hydrate and anhydrate forms of 7-[2-(2-aminothiazol-4-yl)-2-hydroxyiminoacetamide]-3-vinyl-3-cephem-4-carboxylic acid (syn isomer), methods for their preparation, and pharmaceutical compositions comprising these forms.

The present application claims priority to U.S. Provisional Application Ser. No. 60/553,643, filed on Mar. 16, 2004, hereby incorporated in its entirety by reference.

### TECHNICAL FIELD

The present invention relates to trihemihydrate, anhydrate and novel lower hydrate forms of 7-[2-(2-aminothiazol-4-yl)-2-hydroxyiminoacetamide]-3-vinyl-3-cephem-4-carboxylic acid (syn isomer), methods for their preparation, and pharmaceutical compositions comprising the novel forms.

### BACKGROUND OF THE INVENTION

The antimicrobial agent 7-[2-(2-aminothiazol-4-yl)-2-hydroxyiminoacetamido]-3-vinyl-3-cephem-4-carboxylic acid (syn isomer) (hereinafter referred to as “Cefdinir”) is a semi-synthetic oral antibiotic in the cephalosporin family. Cefdinir is sold in the United States as Omnicef® in capsule and oral suspension forms. Omnicef® is active against a wide spectrum of bacteria, including Staphylococcus aureus, Streptococcus pneumoniae, Streptococcus pyogenes, Hemophilus influenzae, Moraxella catarrhalis, E. coli, Klebsiella, and Proteus mirabilis. The preparation of Cefdinir was first disclosed in U.S. Pat. No. 4,559,334, issued Dec. 17, 1985, while the preparation of the commercially available form of Cefdinir (Crystal A or Form I) was first disclosed in U.S. Pat. No. 4,935,507, issued Jun. 19, 1990, both of which are hereby incorporated by reference in their entirety.

Hydrates are important classes of pharmaceutical solids with different chemical and thermodynamic stability. These properties are important criteria when selecting pharmaceutical forms of a compound.

The present invention provides trihemihydrate, anhydrate and novel lower hydrate forms of Cefdinir as well as pharmaceutical compositions and uses thereof. Pharmaceutical compositions comprising these forms of cefdinir and their salts and esters are useful in treating bacterial infections such as Streptococcus pneumoniae and Hemophilus influenzae.

### BRIEF DESCRIPTION OF THE FIGURES

FIG. 1 is the single crystal X-ray diffraction pattern of a trihemihydrate form of cefdinir.

FIG. 2 is the powder X-ray diffraction pattern of a trihemihydrate form of cefdinir.

FIG. 3 is the single crystal X-ray diffraction pattern of a lower hydrate form of cefdinir.

FIG. 4 is the powder X-ray diffraction pattern of a lower hydrate form of cefdinir.

FIG. 5 is the powder X-ray diffraction pattern of anhydrate cefdinir.

FIG. 6 shows two powder X-ray diffraction patterns of two lower hydrate forms of cefdinir.

FIG. 7 is the DMSG analysis showing the Desorption Isotherm of Cefdinir hydrates.

### SUMMARY OF THE INVENTION

The present invention describes trihemihydrate, anhydrate, and other iso-structural lower hydrate forms of Cefdinir.

In one embodiment the present invention describes a novel trihemihydrate crystal form of Cefdinir with 3.5 moles of water per molecule of Cefdinir (approximately 14% by weight of water), with a characteristic peak in the powder X-ray diffraction pattern (PXRD pattern, hereinafter) at a value of two theta of 5.4±0.1°.

In another embodiment the present invention describes a novel trihemihydrate crystal form of Cefdinir with 3.5 moles of water per molecule of Cefdinir (approximately 14% by weight of water), with a characteristic peak in the PXRD pattern at a value of two theta of 10.7±0.1°,

In another embodiment the present invention describes a novel trihemihydrate crystal form of Cefdinir with 3.5 moles of water per molecule of Cefdinir (approximately 14% by weight of water), with a characteristic peak in the PXRD pattern at a value of two theta of 14.2±0.1°.

In another embodiment the present invention describes a novel trihemihydrate crystal form of Cefdinir with 3.5 moles of water per molecule of Cefdinir (approximately 14% by weight of water), with a characteristic peak in the PXRD pattern at a value of two theta of 15.2±0.1°.

In another embodiment the present invention describes a novel trihemihydrate crystal form of Cefdinir with 3.5 moles of water per molecule of Cefdinir (approximately 14% by weight of water), with a characteristic peak in the PXRD pattern at a value of two theta of 21.4±0.1°.

In another embodiment the present invention describes a novel trihemihydrate crystal form of Cefdinir with 3.5 moles of water per molecule of Cefdinir (approximately 14% by weight of water), with a characteristic peak in the PXRD pattern at a value of two theta of 29.2±0.1°.

In another embodiment the present invention describes a novel trihemihydrate crystal form of Cefdinir with 3.5 moles of water per molecule of Cefdinir (approximately 14% by weight of water), with a characteristic peak in the PXRD pattern at a value of two theta of and 30.6±0.1°.

In yet another embodiment the present invention describes a novel trihemihydrate crystal form of Cefdinir with 3.5 moles of water per molecule of Cefdinir (approximately 14% by weight of water), and characteristic peaks in the PXRD pattern at values of two theta of 5.4±0.1°, 10.7±0.1°, 14.2±0.1°, 15.2±0.1°, 21.4±0.1°, 29.2±0.1°.

In another embodiment the present invention describes isostructural lower hydrate crystal forms of Cefdinir with a content of water from 1.7% to 6.1% of water by weight. A lower hydrate of the present invention has a characteristic peak in the PXRD pattern at a value of two theta of 6.0±0.1°.

In another embodiment the present invention describes a lower hydrate with a characteristic peak in the PXRD pattern at a value of two theta of 8.0±0.1°.

In another embodiment the present invention describes a lower hydrate with a characteristic peak in the PXRD pattern at a value of two theta of 11.9±0.1°.

In another embodiment the present invention describes a lower hydrate with a characteristic peak in the PXRD pattern at a value of two theta of 15.9±0.1°.

In another embodiment the present invention describes a lower hydrate which has a characteristic peak in the PXRD pattern at a value of two theta of 16.4±0.1°.

In another embodiment the present invention describes a lower hydrate with a characteristic peak in the PXRD pattern at a value of two theta of 22.4±0.1°.

In another embodiment the present invention describes a lower hydrate with a characteristic peak in the PXRD pattern at a value of two theta of 23.0±0.1°.

In another embodiment the present invention describes a lower hydrate with 1.7% to 6.1% of water by weight which has characteristic peaks in the PXRD pattern at values of two theta of 6.0±0.1°, 8.0±+0.1°, 11.9±0.1°, 15.9±0.1°, 16.4±0.1°, 22.4±0.1°, and 23.0±0.1°.

In yet another embodiment the present invention describes a novel anhydrate crystal form of Cefdinir with a characteristic peak in the PXRD pattern at a value of two theta of 5.5±0.1°.

In another embodiment the present invention describes a novel anhydrate crystal form of Cefdinir with a characteristic peak in the PXRD pattern at a value of two theta of 10.9±0.1°.

In yet another embodiment the present invention describes a novel anhydrate crystal form of Cefdinir with a characteristic peak in the PXRD pattern at a value of two theta of 12.6±0.10.

In yet another embodiment the present invention describes a novel anhydrate crystal form of Cefdinir with a characteristic peak in the PXRD pattern at a value of two theta of 14.7±0.1°.

In yet another embodiment the present invention describes a novel anhydrate crystal form of Cefdinir with a characteristic peak in the PXRD pattern at value of two theta of 16.6±0.1°.

In another embodiment the present invention describes a novel anhydrate crystal form of Cefdinir with a characteristic peak in the PXRD pattern at a value of two theta of 21.8±0.1°.

In another embodiment the present invention describes a novel anhydrate crystal form of Cefdinir with a characteristic peak in the PXRD pattern at value of two theta of 27.3±0.1°.

In yet another embodiment the present invention describes a novel anhydrate crystal form of Cefdinir with characteristic peaks in the PXRD pattern at values of two theta of 5.5±0.1°, 10.9±0.1°, 12.6±0.1°, 14.7±0.1°, 16.6±0.1°, 21.8±0.1+, and 27.3±0.1°.

Another embodiment of the present invention relates to a pharmaceutical composition comprising the trihemihydrate form of Cefdinir of the present invention in combination with a pharmaceutically acceptable carrier.

In yet another embodiment, the present invention relates to a pharmaceutical composition comprising any of the lower hydrate forms of Cefdinir of the present invention in combination with a pharmaceutically acceptable carrier.

In another embodiment, the present invention relates to a pharmaceutical composition comprising the anhydrate form of Cefdinir of the present invention in combination with a pharmaceutically acceptable carrier.

Other embodiments relate to a method for treating bacterial infections by administering any of the pharmaceutical compositions of the present invention.

### DETAILED DESCRIPTION OF THE INVENTION

The present invention relates to a hydrate form of Cefdinir, such as trihemihydrate, an anhydrate form of Cefdinir, and isostructural lower hydrate forms of Cefdinir.

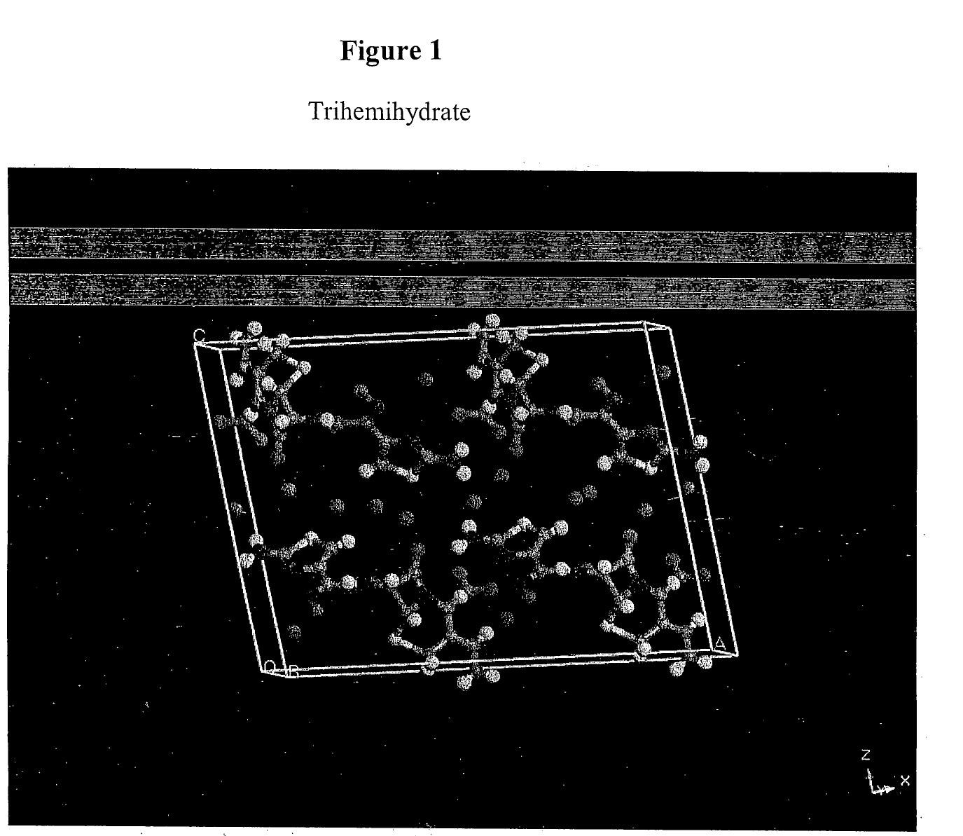

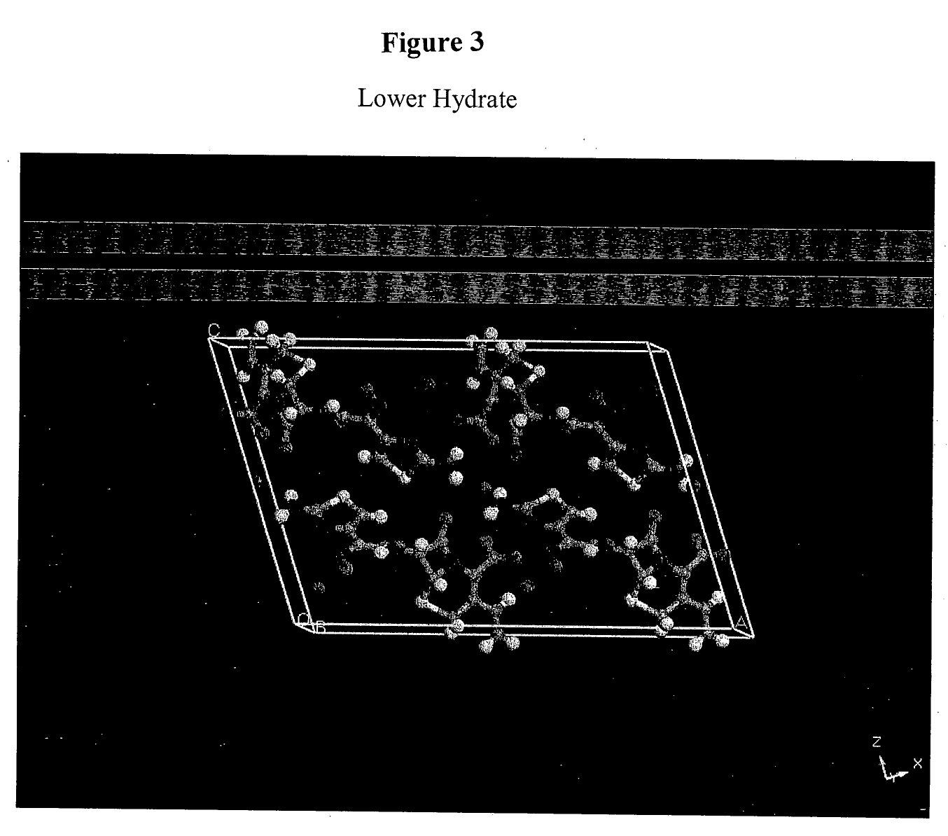

In general, crystalline organic substances contain different amounts of solvent within their crystalline lattice. As used herein hydrates are defined as crystalline forms of an organic substance in which the solvent is water. Hydrates and the anhydrous crystalline forms are characterized by their X-ray diffraction patterns as measured by PXRD and single crystal X-ray Diffraction. Hydrates may solvate or desolvate to form other hydrates. FIG. 1 is the single crystal X-ray Diffraction for the trihemihydrate form of Cefdinir. For four molecules of Cefdinir (large structures) there are 14 molecules of water within the lattice (single dots), representing a 3.5 moles of water per molecule of Cefdinir). It was unexpectedly found that Cefdinir also exists in several lower hydrate forms that despite significant variations in their molar content of water maintain the same PXRD pattern. These low hydrate forms are also called isostructural or isomorphic hydrates because they retain the three-dimensional order of the original crystal, as defined by space group symmetry and the lattice parameters, but have variable amounts of water in the lattice. FIG. 3 is the single crystal X-ray Diffraction for one of this isostructural lower hydrates, which shows that for four molecules of Cefdinir (large structures) there are 5 molecules of water within the lattice (single dots), representing 0.8 moles of water per molecule of Cefdinir.

PXRD was performed on samples of Cefdinir using an XDS-2000/X-ray diffractometer equipped with a 2 kW normal focus X-ray tube and a Peltier cooled germanium solid-state detector (Scintag Inc., Sunnyvale, Calif.). The data was processed using DMSNT software (version 1.37). The X-ray source was a copper filament operated at 45 kV and 40 mA. The alignment of the goniometer was checked daily using a Corundum standard. The sample was placed in a thin layer onto a zero background plate, and continuously scanned at a rate of 2° two-theta per minute over a range of 2 to 40° two-theta.

Characteristic PXRD pattern peak positions are reported in terms of the angular positions (two theta) with an allowable variability of ±0.1°. This allowable variability is specified by the U.S. Pharmacopeia, pages 1843-1884 (1995). The variability of ±0.1° is intended to be used when comparing two powder X-ray diffraction patterns. In practice, if a diffraction pattern peak from one pattern is assigned a range of angular positions (two theta) which is the measured peak position ±0.1° and if those ranges of peak positions overlap, then the two peaks are considered to have the same angular position (two theta). For example, if a diffraction pattern peak from one pattern is determined to have a peak position of 5.2°, for comparison purposes the allowable variability allows the peak to be assigned a position in the range of 5.1°-5.3°. If a comparison peak from the other diffraction pattern is determined to have a peak position of 5.3°, for comparison purposes the allowable variability allows the peak to be assigned a position in the range of 5.2°-5.4°. Because there is overlap between the two ranges of peak positions (i.e., 5.1°-5.3° and 5.2°-5.4°) the two peaks being compared are considered to have the same angular position (two theta).

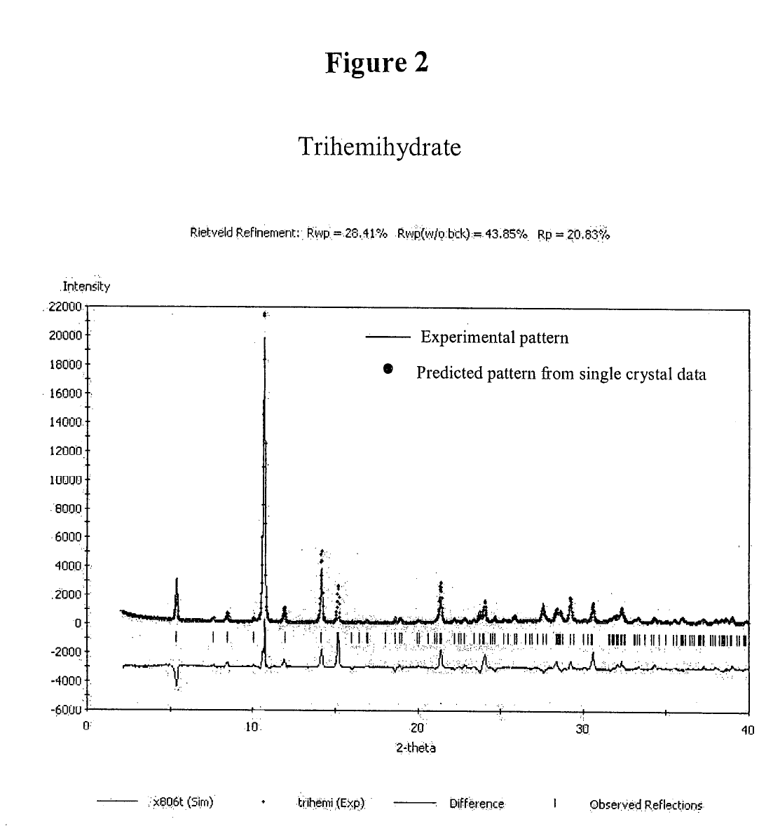

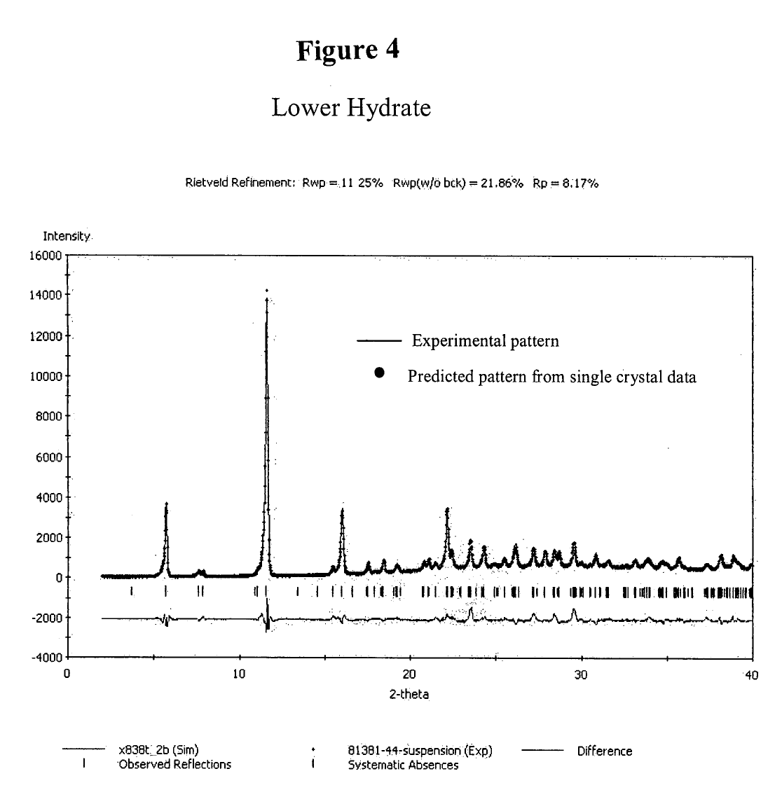

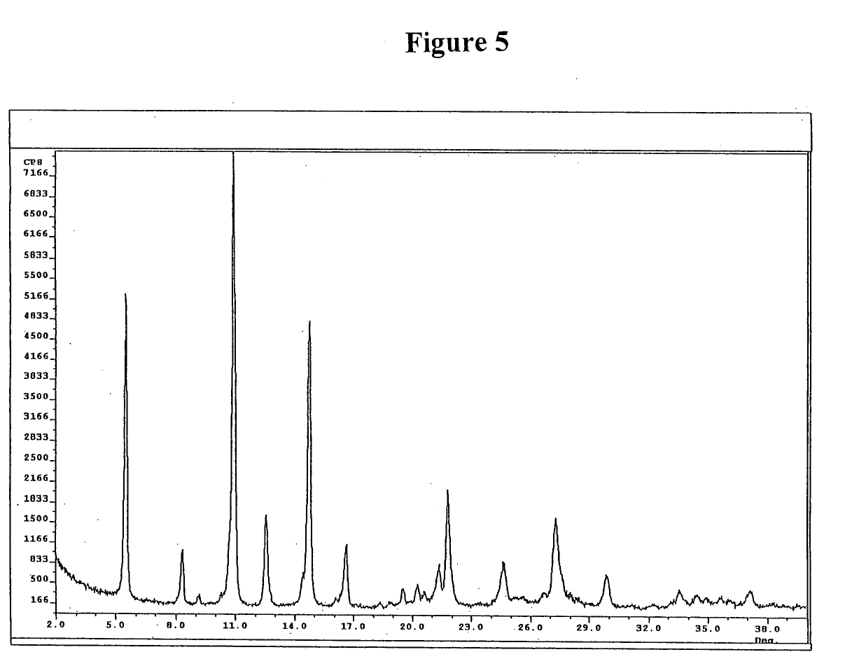

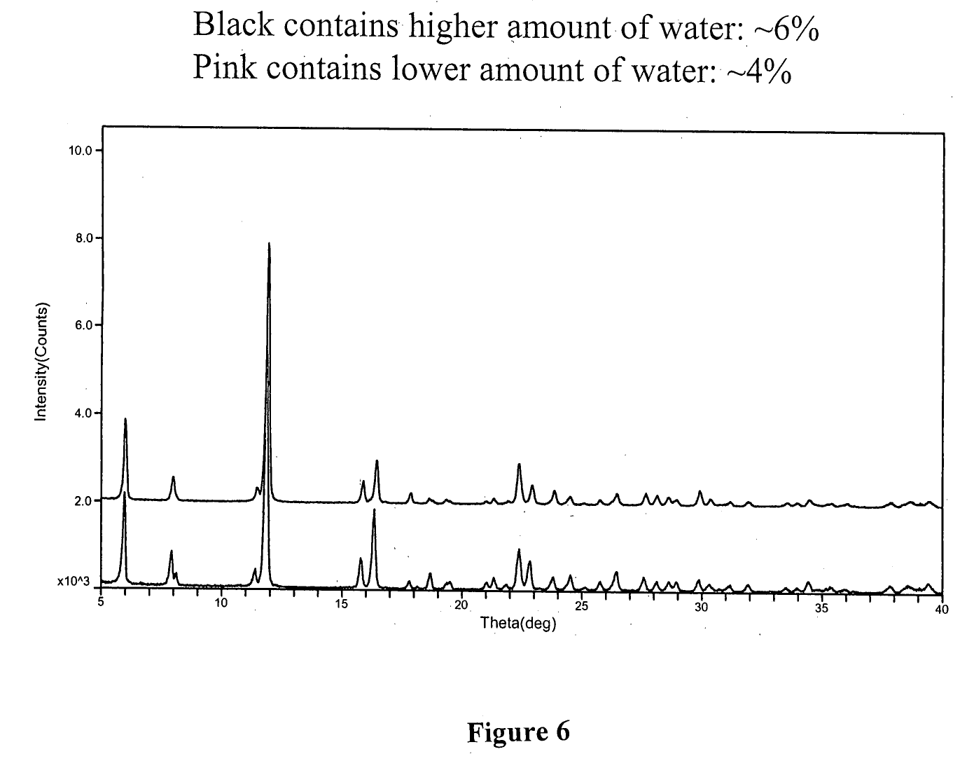

FIGS. 2, 4 and 5 show the different PXRD patterns of the trihemihydrate, an isostructural lower hydrate, and the anhydrate forms of Cefdinir, respectively. As shown in FIG. 2, the trihemihydrate crystal form of Cefdinir, which contains 3.5 moles of water for each molecule of Cefdinir (approximately 14% by weight of water) shows characteristic peaks in the PXRD pattern at values of two theta of 5.4±0.1°, 10.7±0.1°, 14.2±0.1°, 15.2±0.1°, 21.4±0.1°, 29.2±0.1°, and 30.6±0.1°. The upper line represent the predicted pattern obtained from single crystal data and the lower line is the experimental pattern. FIG. 4 shows the isostructural lower hydrate that has characteristic peaks in the PXRD pattern at values of two theta of 6.0±0.1°, 8.0±0.1°, 11.9±0.1°, 15.9±0.1°, 16.4±0.1°, 22.4±0.1°, and 23.0±0.1°. The upper line the predicted pattern obtained from single crystal data and the lower line is the experimental pattern, as for the trihemihydrate, the predicted pattern matches well with the pattern obtained experimentally. As discussed above, these isostructural lower hydrates have different contents of water, from 1.7% to 6.1% by weight, but maintain similar powder X-ray diffraction patterns. FIG. 6 shows the similarity between the PXRD patterns obtained from two of the isostructural lower hydrates of the present invention, one with around 6% of water and another with around 4% of water (1.5 and 0.8 moles of water per molecule of Cefdinir). A novel anhydrate crystal form of Cefdinir, which contains zero percent of water, shows characteristic peaks in the powder X-ray diffraction pattern at values of two theta of 5.5±0.1°, 10.9±0.1°, 12.6±0.1°, 14.7±0.1°, 16.6±0.1°, 21.8±0.1°, and 27.3±0.1° (FIG. 5).

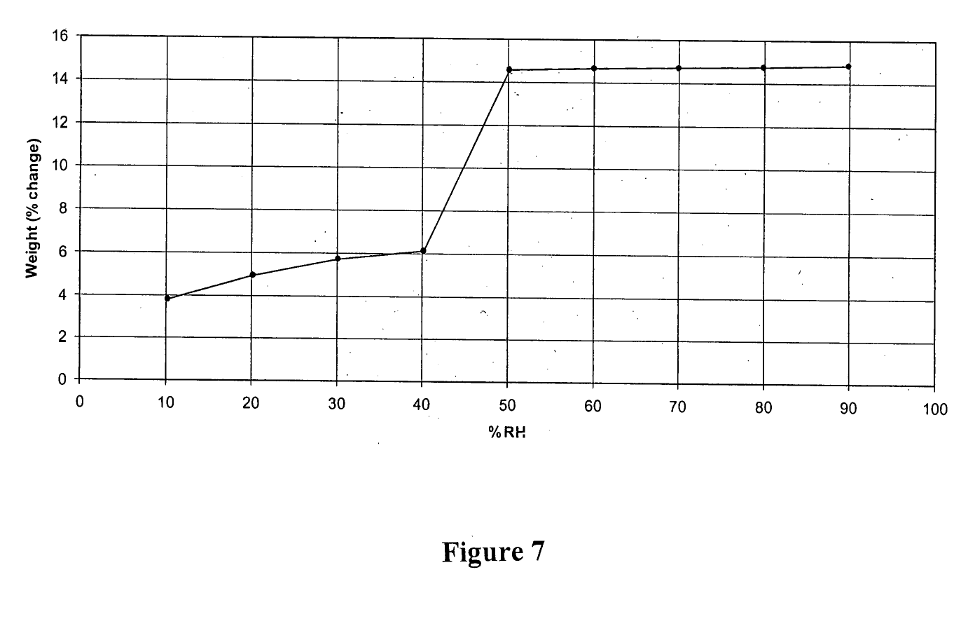

Dynamic Moisture Sorption/Desorption Gravimetric analysis (DMSG hereinafter) was performed for the isostructural lower hydrates. A vacuum moisture balance (MB 300G, VTI Corporation) was used to study the moisture sorption and desoprtion. Samples were first dried at 50° C. under vacuum to a constant weight. The relative humidity was increased to 90% in 10% increments. If the sample weight remained unchanged (i.e. changed by ≦3 mg/15 min), the moisture content was recorded. The balance was calibrated before the experiment and the accuracy of the relative humidity measurement was verified with polyvinylpyrrolidone K90. FIG. 7 shows the moisture desorption isotherm of the hydrates of the present invention. Sharp steps, for example with relative humidity changes from 40% to 50%, occur when the crystal undergoes phase change, i.e. a crystalline structure change. Relatively, flat regions represent a unique phase, i.e. where the crystalline structure does not change and is more physically stable. Increases in the relative humidity from 10% to almost 40%, results in a series of lower hydrate forms of Cefdinir. The novel lower hydrate forms, which are the subject of the present invention, varied but maintained the same crystalline structure and PXRD patterns (see FIG. 6). An increase in the relative humidity from 40% to 50% induced a crystalline structure change, and further increases of the relative humidity from 50% to 90% induced the formation of a more stable phase of the crystal corresponding to a trihemihydrate form of Cefdinir containing approximately 14% by weight of water.

Table 1 summarizes the weight changes of the different hydrate forms of Cefdinir relative to changes in relative humidity. The weight changes are expressed by percentage of water content and by the calculated theoretical molar content of water.

TABLE 1

| % Relative | % of water by | Calculated moles | |
|---|---|---|---|
| Humidity | weight | of water | Hydrate |
| 80.07 | 14.33 | 3.67 | Trihemihydrate |
| 89.90 | 14.80 | 3.81 | Trihemihydrate |
| 79.94 | 14.73 | 3.79 | Trihemihydrate |
| 70.00 | 14.68 | 3.77 | Trihemihydrate |
| 60.10 | 14.63 | 3.76 | Trihemihydrate |
| 50.08 | 14.53 | 3.73 | Trihemihydrate |
| 40.19 | 6.13 | 1.43 | Lower hydrate |
| 30.17 | 5.71 | 1.33 | Lower hydrate |
| 20.24 | 4.94 | 1.14 | Lower hydrate |
| 10.24 | 3.80 | 0.87 | Lower hydrate |

Pharmaceutical Compositions

In accordance with methods of treatment and pharmaceutical compositions of the invention, the compounds can be administered alone or in combination with other agents. When using the compounds, the specific therapeutically effective dose level for any particular patient will depend upon factors such as the disorder being treated and the severity of the disorder; the activity of the particular compound used; the specific composition employed; the age, body weight, general health, sex, and diet of the patient; the time of administration; the route of administration; the rate of excretion of the compound employed; the duration of treatment; and drugs used in combination with or coincidently with the compound used. The compounds can be administered orally, parenterally, intranasally, rectally, vaginally, or topically in unit dosage formulations containing carriers, adjuvants, diluents, vehicles, or combinations thereof. The term “parenteral” includes infusion as well as subcutaneous, intravenous, intramuscular, and intrastemal injection.

Parenterally administered aqueous or oleaginous suspensions of the compounds can be formulated with dispersing, wetting, or suspending agents. The present invention appreciates that the solid forms of the present invention; e.g.: the trihemihydrate and the isostructural lower hydrates can be formulated into suspension products. The injectable preparation can also be an injectable solution or suspension in a diluent or solvent. Among the acceptable diluents or solvents employed are water, saline, Ringer's solution, buffers, monoglycerides, diglycerides, fatty acids such as oleic acid, and fixed oils such as monoglycerides or diglycerides.

The effect of parenterally administered compounds can be prolonged by slowing their release rates. One way to slow the release rate of a particular compound is administering injectable depot forms comprising suspensions of poorly soluble crystalline or otherwise water-insoluble forms of the compound. The release rate of the compound is dependent on its dissolution rate, which in turn, is dependent on its physical state. Another way to slow the release rate of a particular compound is administering injectable depot forms comprising the compound as an oleaginous solution or suspension. Yet another way to slow the release rate of a particular compound is administering injectable depot forms comprising microcapsule matrices of the compound trapped within liposomes, or biodegradable polymers such as polylactide-polyglycolide, polyorthoesters or polyanhydrides. Depending on the ratio of drug to polymer and the composition of the polymer, the rate of drug release can be controlled.

Transdermal patches can also provide controlled delivery of the compounds. The rate of release can be slowed by using rate controlling membranes or by trapping the compound within a polymer matrix or gel. Conversely, absorption enhancers can be used to increase absorption.

Solid dosage forms for oral administration include capsules, tablets, pills, powders, and granules. In these solid dosage forms, the active compound can optionally comprise excipients such as sucrose, lactose, starch, microcrystalline cellulose, mannitol, talc, silicon dioxide, polyvinylpyrrolidone, sodium starch glycolate, magnesium stearate, etc. Capsules, tablets and pills can also comprise buffering agents, and tablets and pills can be prepared with enteric coatings or other release-controlling coatings. Powders and sprays can also contain excipients such as talc, silicon dioxide, sucrose, lactose, starch, or mixtures thereof. Sprays can additionally contain customary propellants such as chlorofluorohydrocarbons or substitutes thereof.

Liquid dosage forms for oral administration include emulsions, microemulsions, solutions, suspensions, syrups, and elixirs comprising inert diluents such as water. These compositions can also comprise adjuvants such as wetting, emulsifying, suspending, sweetening, flavoring, and perfuming agents. Liquid dosage forms may also be contained within soft elastic capsules.

Topical dosage forms include ointments, pastes, creams, lotions, gels, powders, solutions, sprays, inhalants, and transdermal patches. The compound is mixed, if necessary under sterile conditions, with a carrier and any needed preservatives or buffers. These dosage forms can also include excipients such as animal and vegetable fats, oils, waxes, paraffins, starch, tragacanth, cellulose derivatives, polyethylene glycols, silicones, bentonites, talc and zinc oxide, or mixtures thereof. Suppositories for rectal or vaginal administration can be prepared by mixing the compounds with a suitable non-irritating excipient such as cocoa butter or polyethylene glycol, each of which is solid at ordinary temperature but fluid in the rectum or vagina. Ophthalmic formulations comprising eye drops, eye ointments, powders, and solutions are also contemplated as being within the scope of this invention.

Form I of Cefdinir

A pure Cefdinir can be obtained by acidifying the solution containing Cefdinir at room temperature or under warming and thereby having the crystals separate out of the solution.

Suitable examples of the solution containing Cefdinir may include, for example, an aqueous solution of the alkali metal salt of Cefdinir. The solution containing Cefdinir is acidified, if necessary, after said solution is subjected to a column chromatography on activated charcoal, nonionic adsorption resin, alumina, acidic aluminium oxide. The acidifying process can be carried out by adding an acid such as hydrochloric acid or the like preferably in the temperature range from room temperature to 40° C., more preferably, from 15° to 40° C. The amount of the acid to be added preferably makes the pH value of the solution from about 1 to about 4.

A pure Cefdinir can be also obtained by dissolving the Cefdinir in an alcohol (preferably methanol), continuing to stir this solution slowly under warming (preferably below 40° C.), preferably after the addition of water warmed at almost the same temperature as that of said solution, then cooling this solution to room temperature and allowing it to stand.

During the crystallization of Cefdinir, it is preferable to keep the amount slightly beyond the saturation. Cefdinir obtained according to aforesaid process can be collected by filtration and dried by means of the conventional methods.

7-[2-(2-Aminothiazol-4-yl)-2-hydroxyminoacetamido]-3-vinyl-3 -cephem-4-carboxylic acid (syn isomer) (29.55 g) can be added to water (300 ml) and the mixture adjusted to pH 6.0 with saturated sodium bicarbonate aqueous solution. The resultant solution can be subjected to a column chromatography on activated charcoal and eluted with 20% aqueous acetone. The fractions are combined and concentrated to a volume of 500 ml. The resultant solution pH is adjusted to 1.8 at 35° C. with 4N hydrochloric acid. The resultant precipitates are collected by filtration, washed with water and dried to give 7-[2-(2 aminothiazol-4-yl)-2-hydroxyminoacetamido]-3-vinyl-3-cephem-4-carboxylic acid (syn isomer).

Alternatively, to a solution of 7-[2-(2-aminothiazol-4-yl)-2-hydroxyminoacetamido]-3-vinyl-3-cephem-4-carboxylic acid (syn isomer) (0.5 g) in methanol (10 ml) can be added dropwise warm water (35° C.; 1.5 ml) at 35° C. and the resultant solution stirred slowly for 3 minutes, then allowed to stand at room temperature. The resultant crystals are collected by filtration, washed with water and then dried to give 7-[2(2-3-aminothiazol-4-yl)-2-hydroxyminioacetamido]3-vinyl-3-cephem-4-carboxylic acid (syn isomer) as crystals.

The trihemihydrate form of Cefdinir was prepared by suspending Cefdinir, (c.a. 0.8 g) in 1:1 ethanol:ethylacetate solution (a 5 mL beaker was used). To this suspension, approximately 6 drops of concentrated H₂SO₄ was added with intermittent sonication. The solution first turned clear and then a thick yellowish gel was formed. To the gel a couple of drops of water was added and the gel was transferred to the funnel and an attempt to wash the gel resulted in the formation of a white suspension. The white suspension was transferred to centrifuge tubes and centrifuged. The two phases were separated. The aqueous layer discarded, more water was added, vortex mixed and centrifuged. This procedure was repeated until the pH of the aqueous layer was about 3.5. The solid was then analyzed.

Another method to make the trihemihydrate form is to suspend Cefdinir, c.a. 0.8 g in 1:1 ethanol:ethylacetate solution (a 5 mL beaker was used). To this suspension, approximately 6 drops of concentrated H₂SO₄ was added with intermittent sonication. The solution first turned clear and then a thick yellowish gel was formed. To the gel a couple of drops of water was added and the gel was transferred to centrifuge tubes as follows: To each 14 mL tube, 9 mL water was added, then sufficient gel was added to make 12 mL and 2 mL of water added to give 14 mL. Six such tubes were prepared. In each tube white suspension was formed. The white suspension was centrifuged. The two phases were separated. The aqueous layer discarded, more water was added, vortex mixed and centrifuged. This procedure was repeated until the pH of the aqueous layer was about 3.5. The solid was then analyzed.

Lower hydrate forms of Cefdinir were generated by heating the trihemihydrate at 75° C. for 30 min, or by air drying during 3-24 hours, depending on the sample size.

The foregoing is merely illustrative of the invention and is not intended to limit the invention to the disclosed embodiments. Variations and changes, which are obvious to one skilled in the art, are intended to be within the scope and nature of the invention, which are defined, in the appended claims.

1. A trihemihydrate crystal form of Cefdinir with a characteristic peak in the powder X-ray diffraction pattern at value of two theta of 5.4±0.1°.

2. A trihemihydrate crystal form of Cefdinir with a characteristic peak in the powder X-ray diffraction pattern at value of two theta of 10.7±0.1°.

3. A trihemihydrate crystal form of Cefdinir with a characteristic peak in the powder X-ray diffraction pattern at value of two theta of 14.2±0.1°.

4. A trihemihydrate crystal form of Cefdinir with a characteristic peak in the powder X-ray diffraction pattern at value of two theta of 15.2±0.1°.

5. A trihemihydrate crystal form of Cefdinir with a characteristic peak in the powder X-ray diffraction pattern at value of two theta of 21.4±0.1°.

6. A trihemihydrate crystal form of Cefdinir with a characteristic peak in the powder X-ray diffraction pattern at value of two theta of 29.2±0.1°.

7. A trihemihydrate crystal form of Cefdinir with a characteristic peak in the powder X-ray diffraction pattern at value of two theta of 30.6±0.1°.

8. A trihemihydrate crystal form of Cefdinir with characteristic peaks in the powder X-ray diffraction pattern at values of two theta of 5.4±0.1°, 10.7±0.1°, 14.2±0.1°, 15.2±0.1°, 21.4±0.1°, 29.2±0.1°, and 30.6±0.1°.

9. The crystalline form of claim 8, which contains 3.5 moles of water per molecule of Cefdinir.

10. The crystalline form of claim 8, which content of water is 14% by weight.

11. A lower hydrate form of Cefdinir with a characteristic peak in the powder X-ray diffraction pattern at value of two theta of 6.0±0.1°.

12. A lower hydrate form of Cefdinir with a characteristic peak in the powder X-ray diffraction pattern at value of two theta of 8.0±0.1°.

13. A lower hydrate form of Cefdinir with a characteristic peak in the powder X-ray diffraction pattern at value of two theta of 11.9±0.1°.

14. A lower hydrate form of Cefdinir with a characteristic peak in the powder X-ray diffraction pattern at value of two theta of 15.9±0.1°.

15. A lower hydrate form of Cefdinir with a characteristic peak in the powder X-ray diffraction pattern at value of two theta of 22.4±0.1°.

16. A lower hydrate form of Cefdinir with a characteristic peak in the powder X-ray diffraction pattern at value of two theta of 23.0±0.1°.

17. Lower hydrate forms of Cefdinir with characteristic peaks in the powder X-ray diffraction pattern at values of two theta of 6.0±0.1°, 8.0±0.1°, 11.9±0.1°, 15.9±0.1°, 16.4±0.1°, 22.4±0.1°, and 23.0±0.1°.

18. The lower hydrate forms of claim 17, which content of water is 6.1% by weight.

19. The lower hydrate forms of claim 17, which content of water is 6.0% by weight.

20. The lower hydrate forms of claim 17, which content of water is 5.8% by weight.

21. The lower hydrate forms of claim 17, which content of water is 5.7% by weight.

22. The lower hydrate forms of claim 17, which content of water is 5.5% by weight.

23. The lower hydrate forms of claim 17, which content of water is 4.9% by weight.

24. The lower hydrate forms of claim 17, which content of water is 4.4% by weight.

25. The lower hydrate forms of claim 17, which content of water is 3.8% by weight.

26. The lower hydrate forms of claim 17, which content of water is 1.7% by weight.

27. An anhydrate form of Cefdinir with a characteristic peak in the powder X-ray diffraction pattern at value of two theta of 5.5±0.1°.

28. An anhydrate form of Cefdinir with a characteristic peak in the powder X-ray diffraction pattern at value of two theta of 10.9±0.1°.

29. An anhydrate form of Cefdinir with a characteristic peak in the powder X-ray diffraction pattern at value of two theta of 12.6±0.1°.

30. An anhydrate form of Cefdinir with a characteristic peak in the powder X-ray diffraction pattern at value of two theta of 14.7±0.1°.

31. An anhydrate form of Cefdinir with a characteristic peak in the powder X-ray diffraction pattern at value of two theta of 16.6±0.1°.

32. An anhydrate form of Cefdinir with a characteristic peak in the powder X-ray diffraction pattern at value of two theta of 21.8±0.1°.

33. An anhydrate form of Cefdinir with a characteristic peak in the powder X-ray diffraction pattern at value of two theta of 27.3±0.1°.

34. An anhydrate form of Cefdinir with characteristic peaks in the powder X-ray diffraction pattern at values of two theta of 5.5±0.1°, 10.9±0.1°, 12.6±0.1°, 14.7±0.1°, 16.6±0.1°, 21.8+0.1°, and 27.3±0.1°.

35. A pharmaceutical composition comprising the trihemihydrate form of claims 8 or 9 in combination with a pharmaceutically acceptable carrier.

36. A pharmaceutical composition comprising any of the lower hydrate crystal forms of claim 17 in combination with a pharmaceutically acceptable carrier.

37. A pharmaceutical composition comprising the lower hydrate crystal form of claim 18 in combination with a pharmaceutically acceptable carrier.

38. A pharmaceutical composition comprising the lower hydrate crystal form of claim 19 in combination with a pharmaceutically acceptable carrier.

39. A pharmaceutical composition comprising the lower hydrate crystal form of claim 20 in combination with a pharmaceutically acceptable carrier.

40. A pharmaceutical composition comprising the lower hydrate crystal form of claim 21 in combination with a pharmaceutically acceptable carrier.

41. A pharmaceutical composition comprising the lower hydrate crystal form of claim 22 in combination with a pharmaceutically acceptable carrier.

42. A pharmaceutical composition comprising the lower hydrate crystal form of claim 23 in combination with a pharmaceutically acceptable carrier.

43. A pharmaceutical composition comprising the lower hydrate crystal form of claim 24 in combination with a pharmaceutically acceptable carrier.

44. A pharmaceutical composition comprising the lower hydrate crystal form of claim 25 in combination with a pharmaceutically acceptable carrier.

45. A pharmaceutical composition comprising the lower hydrate crystal form of claim 26 in combination with a pharmaceutically acceptable carrier.

46. A pharmaceutical composition comprising the anhydrate crystal form of claim 34 in combination with a pharmaceutically acceptable carrier.

47. A method of treating a bacterial infection by administering a pharmaceutically acceptable composition comprising the crystal form of claim 8.

48. A method of treating a bacterial infection by administering a pharmaceutically acceptable composition comprising the crystal form of claim 9.

49. A method of treating a bacterial infection by administering a pharmaceutically acceptable composition comprising the crystal form of claim 17.

50. A method of treating a bacterial infection by administering a pharmaceutically acceptable composition comprising the crystal form of claim 18.

51. A method of treating a bacterial infection by administering a pharmaceutically acceptable composition comprising the crystal form of claim 19.

52. A method of treating a bacterial infection by administering a pharmaceutically acceptable composition comprising the crystal form of claim 20.

53. A method of treating a bacterial infection by administering a pharmaceutically acceptable composition comprising the crystal form of claim 21.

54. A method of treating a bacterial infection by administering a pharmaceutically acceptable composition comprising the crystal form of claim 22.

55. A method of treating a bacterial infection by administering a pharmaceutically acceptable composition comprising the crystal form of claim 23.

56. A method of treating a bacterial infection by administering a pharmaceutically acceptable composition comprising the crystal form of claim 24.

57. A method of treating a bacterial infection by administering a pharmaceutically acceptable composition comprising the crystal form of claim 25.

58. A method of treating a bacterial infection by administering a pharmaceutically acceptable composition comprising the crystal form of claim 26.

59. A method of treating a bacterial infection by administering a pharmaceutically acceptable composition comprising the crystal form of claim 34.
